(19) United States
(12) Patent Application Publication
(10) Pub. No.: US 2011/0104069 A1
(43) Pub. Date: May 5, 2011

(54) MULTI-FUNCTIONAL BIODEGRADABLE PARTICLES FOR SELECTABLE TARGETING, IMAGING, AND THERAPEUTIC DELIVERY AND USE THEREOF FOR TREATING OCULAR DISORDERS

(75) Inventors: Ronald Xu, Columbus, OH (US); Cynthia Roberts, Columbus, OH (US); Leilei Zhang, Columbus, OH (US)

(73) Assignee: THE OHIO STATE UNIVERSITY, Columbus, OH (US)

(21) Appl. No.: 12/916,065

(22) Filed: Oct. 29, 2010

Related U.S. Application Data
(60) Provisional application No. 61/256,666, filed on Oct. 30, 2009.

(51) Int. Cl. A61K 49/00; A61K 9/14; A61K 39/395; A61P 27/02; A61P 31/00; A61P 29/00; A61P 43/00; B82Y 5/00

(52) U.S. Cl. 424/9.6; 424/490; 424/130.1; 424/491; 424/494; 424/497; 424/133.1; 424/9.1; 977/773; 977/906

(57) ABSTRACT

In various embodiments, provided are multi-functional biodegradable particles for selectable targeting, imaging, and delivery of therapeutic agents. Also provided are methods of using the provided particles for treatment of ocular disorders, such as for the treatment of age-related macular degeneration. The provided particles and methods provide a clinician with options for control over, and monitoring of, the delivery of therapeutic agents.

### CROSS-REFERENCE TO RELATED APPLICATIONS

This patent application claims priority to U.S. Provisional Application No. 61/256,666, filed on Oct. 30, 2009.

### BACKGROUND

Intraocular drug delivery systems have been investigated for treatment of a variety of ocular disorders, including age-related macular degeneration (AMD). Implantable devices and topical delivery systems are well known. However, it is difficult to deliver therapeutic agents to necessary ocular tissues and maintain effective levels of the therapeutic agents using such systems. For example, use of topical formulations for delivery of drugs to the retina typically suffers from poor penetration of the drugs through the blood-retinal barrier of the eye. In light of such limitations, implant systems have been developed. However, implantation of such systems carries a risk of adverse effects, including cataract, retinal detachment, endopthalmitis, and vitreous hemorrhage. Moreover, known implantable systems often suffer from an inability to maintain delivery of drugs at a sufficient level over time to elicit the desired therapeutic response. Accordingly, it is often necessary for multiple implantations to be performed, each carrying with it increased risk of adverse effects. Therefore, there is need in the art for drug delivery systems that are capable of passing the blood-retinal barrier and other limiting ocular tissues, delivering therapeutic agents in a manner that maintains effective levels of the agent over time, and that reduce the risk of adverse effects.

Implantable biodegradable systems have been investigated as one way of meeting this need, as biodegradable systems do not need to be retrieved. For example, microspheres comprised of polylactide (PLA), polyglycolide (PGA), or poly(lactide-co-glycolide) (PLGA) are known as drug delivery systems, and these materials are well characterized as being suitable for intraocular biocompatibility. The mechanical, thermal, and biological properties of such systems may be manipulated to provide a variety of release rates of encapsulated drugs, and because the systems degrade over time, there is reduced risk of adverse effects. Typically, drug release from such systems occurs over three general phases—initial release, extended diffusion, and final release. In the first phase, an initial burst of therapeutic agent occurs at or near the surface of the polymer after implantation, the diffusion rate dependent upon, among other things, surface area of the system, drug loading, and hydrophobicity of the therapeutic agent. In the second phase, the polymer becomes progressively eroded, which creates and increases the number of pores or channels in the polymer, which allows for diffusion of the drug. The rate of release is dependent upon the rate of polymer erosion. In the third phase, the integrity of the polymer becomes compromised through further erosion and degradation, thereby resulting in a second burst of drug. The second burst is largely uncontrollable and is thus, typically undesired.

Known biodegradable implantable delivery systems for intraocular delivery of drugs offer advantages over topical systems and non-biodegradable implants. However, such biodegradable systems are not without limitations. Because such systems are dependent upon rates of polymer degradation for drug delivery, a clinician has little control over drug delivery after implantation and limited ability to monitor the degradation/delivery process. As a consequence, known biodegradable systems may require repeated implantation to achieve an effective drug concentration in the relevant ocular tissue and to maintain it over a prescribed period of time. Repeated implantation of such biodegradable systems carries with it adverse effects. Additionally, adverse effects from the microparticles themselves have been reported, including vitreous clouding, foreign body reaction, and fibrosis. For example, it has been reported that microparticles greater than 5-10 μm in diameter may not be phagocytosed within macrophages and foreign body giant cells, creating a foreign body reaction. It has also been reported that microparticles less than 5 μm in diameter may undergo phagocytosis, making them susceptible to rapid degradation after implantation, which increases the need for repeated implantation.

Although implantable biodegradable ocular drug delivery systems are known, there remains a need for improved systems. In particular, there remains a need for systems that can deliver therapeutic agents in a manner that allows for therapeutic concentration to be maintained over time in relevant ocular tissues. Additionally, there remains a need for delivery systems that provide a clinician with control over the delivery of therapeutic agents. Moreover, there remains a need for systems that provide a clinician with options for real-time monitoring of the systems and therapeutic agent delivery processes.

### SUMMARY

These needs are met by the present application, which provides in various embodiments, multi-functional biodegradable microparticles and nanoparticles for selectable targeting, imaging, and delivery of therapeutic agents for treatment of ocular disorders. Said particles comprise (a) a biodegradable shell; (b) at least one ocular targeting agent coupled to the exterior surface of the shell; and (c) at least one filler agent encapsulated within the shell. The ocular targeting agent may be, but is not required to be, a therapeutic agent. In various embodiments, the particles also comprise at least one therapeutic agent encapsulated within the shell. The provided particles are adapted to migrate to the ocular target after administration and to deliver at least one therapeutic agent at a first rate. The particles are also adapted to expand upon exposure to energy from at least one energy source, said expansion affecting shell integrity and thickness, which allows for optional delivery of at least one encapsulated therapeutic agent at rates greater than the first rate.

Also provided are methods of using the provided microparticles and nanoparticles for treatment of ocular disorders. In some embodiments, said methods relate to treatment of AMD. The provided methods comprise (a) administering to a subject a composition comprising at least one of the provided particles, said particles comprising at least one ocular targeting agent; (b) pausing for a pre-determined period of time to allow the administered particles to migrate to the ocular target; (c) optionally, confirming migration to the ocular target using one or more ocular imaging tools; and (d) optionally, administering sufficient energy from at least one energy source to cause the migrated particles to expand.

The provided particles and methods allow a clinician to selectably control the multiple functions of the particles to deliver therapeutic agents in a controllable process. Delivery of therapeutic agents may optionally be monitored by ocular imaging tools before, during, or after delivery of the therapeutic agents, thereby providing a clinician with additional control over the particles and the delivery process.

These and additional embodiments will become apparent in the course of the following detailed description.

### BRIEF DESCRIPTION OF THE DRAWINGS

A more complete appreciation of the invention and the many embodiments thereof will be readily obtained as the same becomes better understood by reference to the following detailed description as considered in reference to the accompanying drawings, wherein:

FIG. 1 illustrates one exemplary embodiment of the provided particles;

FIG. 2 illustrates a contemplated mechanism of action of the particles of FIG. 1;

FIG. 3 illustrates one exemplary embodiment of the provided particles;

FIG. 4 illustrates one exemplary embodiment of the provided particles;

FIG. 5 illustrates one exemplary embodiment of the provided particles;

FIG. 6 schematically illustrates one exemplary method of conjugating a targeting agent to the shell of a provided particle;

FIG. 7 illustrates one exemplary embodiment of the provided methods for treating ocular disorders; and

FIG. 8 illustrates one exemplary embodiment of the provided methods for image-guided delivery of anti-VEGF therapeutics for the treatment of AMD.

### DETAILED DESCRIPTION

Specific embodiments of the present invention will now be described. The invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the present description is for describing particular embodiments only and is not intended to be limiting of the invention.

As used in the description and the appended claims, the singular forms “a,” “an,” and “the” are intended to include the plural forms as well, unless the context clearly indicates otherwise.

As used in the description and appended claims, and unless the context clearly indicates otherwise, the term “antibody” is intended to mean whole immunoglobulins and immunoglobulin fragment preparations that are reactive with discrete regions of an antigen. The term includes polyclonal antibodies, monoclonal antibodies, antibody fragments, and chimeric versions of whole antibodies or antibody fragments, without regard to origin or source. One example of an antibody is a humanized monoclonal IgG1 or fragment thereof. Antibodies and various methods of preparing them are known.

The term “antigen,” as used in the description and appended claims, includes but is not limited to proteins, peptides, receptors, hormones, carbohydrates, lipids, nucleic acids, and differentiated cells or tumor cells that are capable of eliciting an immunological response that leads to the production of an antibody population.

The term “coupled,” as used in the description and appended claims is intended to mean connected, whether directly or indirectly. Accordingly, the term includes covalent bonds and non-covalent interactions (such as electrostatic forces). The term also includes indirect connections, such as for example, between a polymer surface, an antibody covalently bound to said surface, and a fluorophore covalently bound to said antibody. In such example, the polymer surface and fluorophore could be described as being coupled.

As used in the description and appended claims, and unless the context clearly indicates otherwise, the term “particle,” is intended to refer to spheres, rods, or other shapes having at least one dimension (for example, diameter) that is from 1 to 1000 μm (a “microparticle”), as well as spheres, rods, or other shapes having at least one dimension (for example, diameter) that is from 1 to 1000 nm (a “nanoparticle”). A provided particle may encapsulate materials existing as one phase or existing as more than one phase. The encapsulated materials may also exist as an oil-in-water or water-in-oil emulsions, miniemulsions, or microemulsions. A particle may be adapted such that the surface carries an overall electrostatic charge. While both microparticles and nanoparticles are within the scope of this application, certain embodiments may apply only to one particle type. For example, one type of particle may be useful in targeting one specific ocular target, whereas both types of particles may be useful in targeting another ocular target.

The term “therapeutic agent,” as used in the description and appended claims, is intended to include, but not be limited to, antibodies, proteins, peptides, genes, gene fragments, small molecules, drugs, hormones, compositions, formulations, and other agents that, when delivered, elicit a therapeutic response themselves or aid another agent in eliciting a therapeutic response. Accordingly, a therapeutic agent may be a pharmaceutically active ingredient in a composition or may be an adjuvant. In some embodiments, the provided particles comprise one or more therapeutic agents that are pharmaceutically active.

Unless otherwise indicated, all numbers expressing quantities of ingredients, properties such as molecular weight, reaction conditions, and so forth as used in the specification and claims are to be understood as being modified in all instances by the term “about.” Additionally, the disclosure of any ranges in the specification and claims are to be understood as including the range itself and also anything subsumed therein, as well as endpoints. Unless otherwise indicated, the numerical properties set forth in the specification and claims are approximations that may vary depending on the desired properties sought to be obtained in embodiments of the present invention. Notwithstanding that numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

In various embodiments, provided are multi-functional biodegradable microparticles and nanoparticles for selectable targeting, imaging, and delivery of therapeutic agents for treatment of ocular disorders. It is contemplated that the provided particles may be adapted for placement in subconjunctival, sub-tenon, intrascleral, intravitreal, or subretinal spaces by injection or other method of implantation. In some embodiments, the particles are adapted to be administered by intra-ocular injection. In some embodiments, the provided particles are nanoparticles adapted for intravitreal injection and targeted delivery of therapeutic agents for treatment of AMD. However, it is also contemplated that the provided nanoparticles and microparticles may be adapted for targeted delivery of therapeutic agents for treatment of a variety of other ocular disorders, including but not limited to, glaucoma, infective conjunctivitis, allergic conjunctivitis, ulcerative keratitis, non-ulcerative keratitis, episcleritis, scleritis, diabeticretinopathy, uveitis, endophthalmitis, infectious conditions, and inflammatory conditions. It is also contemplated that the particles may be adapted for delivery of antibiotics, anti-fungal agents, anti-viral agents, or combinations thereof as part of or after an ocular surgical procedure. Examples of such procedures include, but are not limited to, keratoplasty, lamellar procedures, cataract procedures, and retinal detachment procedures.

In the various embodiments, the provided particles are adapted to migrate to an ocular target, which may be one or more specific tissues or substances contained therein (collectively, “targeted tissue”) associated with an ocular disorder. For example, an ocular target may be a protein, a protein receptor, or a cell expressing the protein or protein receptor. Accordingly, the targeted tissue will, at least in part, determine whether the particles selected for a particular application are microparticles or nanoparticles. In an exemplary embodiment, an ocular target may be selected from vascular endothelial growth factor (VEGF), vascular endothelial growth factor receptor 2 (VEGF R2), and retina pigment epithelial cells and the particles selected are nanoparticles which are of appropriate size to penetrate and migrate through openings of the permeable vasculature, settle on the internal limiting membrane and be transported to the retina pigment epithelium. Intraocular location of the provided particles may, in some embodiments, be imaged after administration to a subject, thereby allowing for detection of the particles within targeted tissue. In some embodiments, the provided particles allow for delivery of therapeutic agents in an iterative, controllable process.

In various embodiments, the provided particles comprise (a) a biodegradable shell; (b) at least one ocular targeting agent coupled to the exterior surface of the shell; (c) at least one filler agent encapsulated within the shell; and (d) at least one therapeutic agent encapsulated within the shell. The particles are adapted to migrate to the targeted ocular tissue after administration to the subject (such as by intra-ocular injection) and to deliver the at least one therapeutic agent at a first rate over a first pre-determined period of time. The particles are also adapted to expand upon exposure to energy from at least one energy source, said expansion affecting shell integrity and thickness. This allows for optional delivery of the at least one therapeutic agent at a second rate over a second pre-determined period of time, the second rate greater than the first rate. The second rate can be determined by degree of particle expansion, which can be controlled by selection of energy source, energy strength, and duration of exposure. In some embodiments, exposure to energy from an energy source may occur more than once, thereby allowing for optional delivery of the at least one therapeutic agent at least a third rate over at least a third pre-determined period of time, the third rate greater than the second rate.

In addition to multi-functional biodegradable particles, also provided are methods for treatment of an ocular disorder. Said methods comprise (a) administering to a subject (such as by intra-ocular injection) a composition comprising at least one of the provided particles, said particles comprising at least one ocular targeting agent; (b) pausing for a pre-determined period of time to allow the administered particles to migrate to the targeted ocular tissue; (c) optionally, confirming migration to the targeted ocular tissue using one or more ocular imaging tools; and (d) optionally, administering sufficient energy from at least one energy source to cause the migrated particles to expand. The provided methods allow a clinician to selectably control the multiple functions of the particles and deliver therapeutic agents in an iterative, controllable process. In an exemplary embodiment, the clinician may allow the administered particles to migrate to the targeted ocular tissue and deliver the at least one therapeutic agent at a first rate over a first pre-determined period of time. The particles are adapted to expand upon exposure to energy from an energy source, and the clinician may control degree of particle expansion by selection of energy source, energy strength, and duration of exposure. Accordingly, in another exemplary embodiment, the clinician may optionally control delivery of the at least one therapeutic agent at a second rate over a second pre-determined period of time, the second rate greater than the first rate and determined by degree of particle expansion. Exposure to energy from an energy source may occur more than once. Accordingly, in yet another exemplary embodiment, the clinician may optionally control delivery of the at least one therapeutic agent at a third rate over a third pre-determined period of time, the third rate greater than the second rate and determined by degree of additional particle expansion.

### Particles

Provided are microparticles and nanoparticles comprising (a) a biodegradable shell; (b) at least one ocular targeting agent and optionally, at least one imaging agent, coupled to the exterior surface of the shell; and (c) at least one filler agent encapsulated within the shell. In some embodiments, also encapsulated within the shell is at least one therapeutic agent. The provided particles are adapted to be used for imaging purposes, therapeutic purposes, or a combination thereof. Particles comprising an encapsulated therapeutic agent, a therapeutic targeting agent coupled to the shell, or both, are adapted to treat an ocular disorder by eliciting a therapeutic response or aiding in eliciting a therapeutic response. Particles lacking a therapeutic agent but comprising an encapsulated imaging agent, an imaging agent coupled to the shell, or both, are adapted to diagnose or monitor an ocular disorder, or to monitor delivery of therapeutic agents administered by other means. Particles comprising therapeutic agents and imaging agents are adapted for imaging and therapeutic purposes.

In the various embodiments, the provided particles comprise a biodegradable shell. Biodegradable shells and methods of their preparation are known. In some embodiments, the biodegradable shell of the provided particles may comprise a material selected from lipid, human serum albumin, polylactide (PLA), poly(ε-caprolactone) (PCL), poly(glycolic acid) (PGA), poly(lactide-co-glycolide) (PLGA), chitosan, eudragit, hyaluronic acid, alginate, carboxy methyl cellulose, carbopol, polyethylene glycol, poly(ethyl-2-cyanoacrylate) (PECA), polystyrene, poloxamers (such as Pluronic® block copolymers), Hydroxypropyl Methylcellulose (HPMC), 2-hydroxyethyl methacrylate (HEMA), polyvinyl alcohol (PVA), poly(methyl acrylate) (PMA), and other biodegradable and biocompatible materials for use in implant applications, as well as combinations thereof. In some embodiments, the shell comprises one or more of lipid, human serum albumin, PLA, PGA, and PLGA. Good results have been obtained with PLGA.

As one of skill in the art will appreciate, the biodegradation rate of the shell and therapeutic agent release rates may be controlled by selection and design of the shell material. For example, molecular weight and chemical composition may be selected to achieve the desired particle shell properties, such as biodegradation occurring over a period of from 1 day to 6 months. In some embodiments, electrostatic charge of the particle shell may also be designed to assist in targeting. For example, it may be desirable to have a particle with a negatively charged PLGA shell to aid in the diffusion of the particle through the three-dimensional vitreal network of collagen fibrils in the eye. In addition to control over therapeutic agent release kinetics, biodegradation rate, and intraocular diffusion, use of biodegradable materials for the provided particles also allows the particles to be cleared from the eye after delivery of the at least one therapeutic agent.

The provided particles are adapted to be of a size which permits them to penetrate relevant tissues and migrate to the targeted ocular tissue. For example, some particles are adapted to penetrate through openings of the permeable vasculature, including the internal limiting membrane separating the vitreous chamber and retina of the eye. Particles having various shapes, such as spheres and rods, are contemplated. Good results have been obtained with spheres. In some embodiments, provided are microparticles having a diameter of from about 1 to 1000 μm. Accordingly, the diameter may be from 1-100 μm, 100-200 μm, 200-300 μm, 300-400 μm, 400-500 μm, 500-600 μm, 600-700 μm, 700-800 μm, 800-900 μm, and 900-1000 μm. In some embodiments, provided are nanoparticles having a diameter of from about 1 to about 1000 nm. Accordingly, the diameter may be from 1-100 nm, 100-200 nm, 200-300 nm, 300-400 nm, 400-500 nm, 500-600 nm, 600-700 nm, 700-800 nm, 800-900 nm, and 900-1000 nm. For example, the diameter of nanoparticles adapted to target the retina pigment epithelium may be from 1-600 nm; alternatively, 200-400 nm; alternatively, 300-400 nm.

In the various embodiments, the provided particles comprise an ocular targeting agent coupled to the exterior surface of the shell. The ocular targeting agent may, in some embodiments, be selected from antibodies, aptamers, peptides, lectins, short chain and long chain organic molecules, inorganic molecules, and fragments or derivatives thereof, provided that such targeting agents have an affinity for at least one target within ocular tissue or will have an affinity for the tissue itself. In an exemplary embodiment, the target may be a receptor associated with an ocular disorder or a protein associated with an ocular disorder. Thus, coupling an ocular targeting agent to the shell of the provided particles allows the particles to concentrate at one or more specific locations within ocular tissue associated with an ocular disorder. One having skill in the art will appreciate that selection of a particular targeting agent for conjugation to the particle shell will depend, in part, upon the nature of the ocular disorder of interest and the chosen ocular tissue target. In some embodiments, the targeting agent may be a therapeutic agent. Accordingly, a targeting agent may, in addition to aiding in the delivery of the particle to targeted ocular tissue, provide a therapeutic benefit. In some embodiments, the provided particles lack an encapsulated therapeutic agent but comprise a therapeutic targeting agent coupled to the shell. In some embodiments, the provided particles comprise a therapeutic targeting agent coupled to the shell, as well as at least one encapsulated therapeutic agent.

In some embodiments, the targeting agent may be an antibody coupled to the exterior shell of the provided particles, the antibody specific for an antigen produced by or associated with ocular tissue implicated in an ocular disorder. In an exemplary embodiment, one or more anti-VEGF R2 antibodies (or fragment thereof) may be coupled to the exterior shell of provided nanoparticles, thereby allowing targeting of retina pigment epithelial cells expressing vascular endothelial growth factor receptor 2 (VEGF R2). As another exemplary embodiment, one or more anti-VEGF antibodies (or fragment thereof) may be coupled to the exterior shell of the provided nanoparticles, thereby allowing targeting of vascular endothelial growth factor (VEGF) itself. Over-expression of VEGF and VEGF R2 receptors by retina pigment epithelial cells is associated with AMD, and the provided nanoparticles allow for targeted delivery of anti-VEGF therapeutics. Examples of anti-VEGF R2 targeting agents suitable for conjugation to the shell exterior surface include, but are not limited to, anti-VEGF R2 monoclonal antibodies and heterodimer peptides. Good results have been obtained with anti-VEGFR2 mAb clone Avas12a1 (eBioscience, Inc) and anti-VEGFR2 mAb 2C3 (Peregrine Pharmaceuticals). Examples of anti-VEFG targeting agents suitable for conjugation to the shell exterior include, but are not limited to, bevacizumab and ranibizumab. In an exemplary embodiment, anti-VEGF R2 antibodies and anti-VEGF antibodies may be coupled to the provided nanoparticle shells by activating carboxyl functional groups on the exterior shell surface with 1-ethyl-3-(3-dimethylaminopropyl) carbodiimide hydrochloride (EDC). Using N-hydroxy succinimide (NHS), the activated carboxyl functional groups may then be NHS-ester activated and mixed with the antibodies and stirred for immediate reaction. The exterior shell surface of nanoparticles may also be coupled with antibodies through avidin-biotin chemistry wherein biotin is first coupled with NHS-ester active nanoparticles and then streptavidin is applied, followed by the application of biotinylated antibodies.

The provided particles may, in some embodiments, optionally comprise at least one imaging agent coupled to the external shell surface in addition to the coupled ocular targeting agent. Conjugation of an imaging agent allows for image-guided targeted delivery of therapeutic agents by allowing particle intraocular location to be imaged before, during, or after therapeutic delivery. A variety of imaging agents are suitable for coupling to the shell surface including, but not limited to, fluorescence imaging agents, radionuclide-labeled imaging agents (such as agents comprising iodine-124), and magnetic resonance imaging agents (such as gadolinium contrast agents). In some embodiments, the provided particles comprise a fluorescence imaging agent coupled to the shell surface, said agent detectable by fluorescence imaging. However, more than one type of imaging agent or imaging technique may be used to detect particle intraocular location. For example, fluorescence imaging may be combined with other imaging techniques, such as ultrasound imaging. Because ultrasound imaging particles has a low specificity due to tissue heterogeneity, sensitivity limitation, and image artifacts, conjugation of a fluorescence imaging agent to a particle shell allows for combined use of ultrasound and fluorescence imaging to provide the sensitivity and specificity necessary for quantitative image-guided delivery of therapeutic agents.

In an exemplary embodiment, the provided particles comprise at least one fluorescence imaging agent coupled to the external surface of the shell, thereby allowing for confirmation of particle localization at targeted ocular tissue, determination of particle concentration at the targeted ocular tissue, or both, before expansion of the particle by exposure to a source of energy. Thus, such pre-expansion confirmation, determination, or both may occur prior to beginning or increasing the rate of delivery of the encapsulated therapeutic agents. Moreover, ultrasound imaging may be additionally be used to monitor particle localization, concentration, or both before expansion of the particle, as well as to monitor particle localization, concentration, or both after expansion of the particle.

Fluorescence imaging agents that may be coupled to the shell include, but are not limited to, indocyanine green, cyanine 5, cyanine 7, cyanine 9, Texas Red® (Invitrogen), Nile Red® (Invitrogen), fluorescein, green fluorescent protein, red fluorescent protein, yellow fluorescent protein, IRDye™ 800CW (Li-Cor Biosciences), near infrared fluorescence type II quantum dots, Accudrop™ fluorescent beads (BD Biosciences), AlexaFluor 680 (Invitrogen), and combinations thereof. Good results have been achieved with AlexaFluor 680, IRDye™ 800CW, and cyanine 7. Conjugation of the fluorescence agent to the shell surface may be achieved by a process similar to conjugation of the ocular tissue targeting agent. In one exemplary embodiment, the NHS-ester active particles may be coupled with biotin in a 2-(N-morpholino)ethanesulfonic acid (MES) buffer solution, reacted with streptavidin, followed by the reaction with a biotinylated Cyanine 7 that is prepared using commercially available biotinylation kits.

In the various embodiments, the provided particles comprise at least one filler agent encapsulated within the shell. In some embodiments, the filler agent may be selected from air, perfluorocarbon (liquid), perfluorocarbon (gas), nitrogen, saline, phosphate buffered saline, water, fluorescence imaging agent, photoacoustic agent, and combinations thereof. More than one filler agent or type may be encapsulated in the provided particles. For example, the particles may comprise one, two, three, or more different filler agents or different types of the same agent. One of skill in the art will appreciate that selection of filler agents for encapsulation depends, in part, upon the desired properties of the particles and intended application. For example, at least one filler agent must be expandable upon exposure to energy from at least one energy source, as expansion of the filler agent will cause expansion of the particle. As another example, if photoacoustic imaging of the expanded particle is desired, then at least one photoacoustic imaging agent would additionally need to be selected as a filler agent. In some embodiments, the provided particles comprise from about 0.1 to about 50% (w/v) of the at least one filler agent. Accordingly, the concentration of one or more filler agents encapsulated in the particle may be 0.1-1%, 1-5%, 5-10%, 10-15%, 15-20%, 20-25%, 25-30%, 30-35%, 35-40%, 40-45%, 45-50% (w/v).

In some embodiments, encapsulated within the provided particles is one or more types of perfluorocarbon. The provided particles may, in some embodiments, comprise from about 0.1 to about 50% (w/v) of perfluorocarbon, which is a heat-sensitive compound that, when heated (such as through ultrasonic pulses of appropriate intensity and duration) can greatly expand. For example, a nanoparticle comprising perfluorocarbon may exhibit a 1-15 fold expansion when heated by administration of appropriate ultrasound energy. Accordingly, expansion of a nanoparticle by from 1-2, 2-3, 3-4, 4-5, 5-6, 6-7, 7-8, 8-9, 9-10, 10-11, 11-12, 12-13, 13-14, or 14-15 times may occur upon administration of energy. In some embodiments, the provided particles may comprise liquid perfluorocarbon and phosphate buffered saline, wherein the therapeutic agent may be loaded into the phosphate buffered saline phase. In some embodiments, the provided particles may comprise perfluorocarbon and an imaging agent, wherein the combination of expanded particles and imaging agent presence enhances the ability to monitor therapeutic dosimetry, particle degradation, or both.

Encapsulated within the provided particles may, in some embodiments, be one or more types of fluorescence imaging agent. In some embodiments, the provided particles may comprise from about 0.1 to about 50% (w/v) of fluorescence imaging agent. Encapsulation of the fluorescence imaging agent aids in protecting it from molecular interactions with the vitreous fluid, and it allows for long-term use of fluorescence imaging to monitor particle degradation and release of therapeutic agents. For example, encapsulating of indocyanine green in particles protects it from molecular interaction with the surrounding tissue environment and produces stabilized absorption and fluorescence spectra for optical imaging, hyperspectral, imaging, photoacoustic imaging, and fluorescence imaging. Fluorescence imaging agents suitable for encapsulation include, but are not limited to, indocyanine green, cyanine 5, cyanine 7, cyanine 9, Texas Red® (Invitrogen), Nile Red® (Invitrogen), fluorescein, green fluorescent protein, red fluorescent protein, yellow fluorescent protein, IRDye™ 800CW (Li-Cor Biosciences), near infrared fluorescence type II quantum dots, Accudrop™ fluorescent beads (BD Biosciences), AlexaFluor 680 (Invitrogen), and combinations thereof. In some embodiments, one or more of indocyanine green, cyanine 5, cyanine 7, cyanine 9, fluorescein, and green fluorescent protein are encapsulated within the provided particles. In some embodiments, the provided particles may comprise at least one perfluorocarbon and at least one fluorescence imaging agent.

In some embodiments, encapsulated within the provided particles may be one or more types of photoacoustic agent. The provided particles may, in some embodiments, comprise from about 0.1 to about 50% (w/v) of photoacoustic agent. Suitable photoacoustic agents include, but are not limited to, India ink, methylene blue, gold nanoparticles, carbon nanotubes, Fiesta Red (Private Reserve Ink), and Rhodamine (Arcos Organics N.V.). In some embodiments, the provided particles may comprise at least one encapsulated perfluorocarbon and at least one encapsulated photoacoustic agent. In some embodiments, the provided particles may comprise at least one encapsulated perfluorocarbon, at least one encapsulated fluorescence imaging agent, and at least one encapsulated photoacoustic agent.

The provided particles may, in some embodiments, comprise various types of the same filler agent, each type providing a different functionality. By mixing filler agents having different properties (such as boiling point) at certain ratios, particles can be designed to have tunable properties which can be exploited for control of targeted delivery of therapeutic agents in the treatment of an ocular disorder. In an exemplary embodiment, encapsulated within a nanoparticle may be more than one type of perfluorocarbon, each type having a different boiling point. For example, n-PFP (boiling point of 29.3° C.) and H2-PFP (boiling point of 53.5° C.) may be encapsulated within the same nanoparticle, said nanoparticle being tuned to expand upon exposure to ultrasound within a predetermined threshold of acoustic intensity and frequency.

In the various embodiments, the provided particles comprise at least one encapsulated therapeutic agent. Accordingly, the particle may comprise one, two, three, or more therapeutic agents for treatment of an ocular disorder. Suitable therapeutic agents include, but are not limited to, bevacizumab (for example, Avastin®, Genentech), ranibizumab (for example, Lucentis®), pegabtanib (for example, Macugen®), oligonucleotides, Acetazolamide, Pilocarpine HCl, Insulin, Cyclopentolate, Timolol maleate, GCV, Pilocarpine, Amikacin, Flurbiprofen, Cyclosporin, Rhodamine, Dexamethasone, Pilocarpine nitrate, tripicamide, antibiotics, antifungal agents, anti-viral agents, and combinations thereof. One of skill in the art will appreciate that the ocular targeting agent coupled to the external surface of the shell may, in some embodiments, be the same as the therapeutic agent encapsulated within the shell. In some embodiments, the encapsulated therapeutic agent may be loaded into at least one filler agent phase. For example, an anti-VEGF antibody may be loaded into a phosphate buffered saline (PBS) phase of a nanoparticle comprising PBS and at least one perfluorocarbon filler agent.

In some embodiments, the provided particles comprise from about 0.1 to about 50% (w/v) of the at least one therapeutic agent. Accordingly, the concentration of one or more therapeutic agents encapsulated in the particle may be 0.1-1%, 1-5%, 5-10%, 10-15%, 15-20%, 20-25%, 25-30%, 30-35%, 35-40%, 40-45%, or 45-50% (w/v). In an exemplary embodiment, provided are nanoparticles that comprise an anti-VEGF therapeutic agent for treatment of AMD selected from bevacizumab, ranibizumab, and pegabtanib, wherein the nanoparticles comprise from about 1 to about 5% (w/v) of the selected anti-VEGF therapeutic agent.

The encapsulated therapeutic agent may, in some embodiments, be freeze-dried or otherwise treated to inhibit or prevent dissolution or diffusion through an unexpanded shell. In an exemplary embodiment, an encapsulated anti-VEGF therapeutic (such as bevacizumab, ranibizumab, or pegabtanib) may be freeze-dried. The shell may be constructed such that diffusion of fluid through the shell does not occur until the particle has been expanded by exposure to energy from at least one energy source. Upon expansion, diffusion may occur, thereby allowing the therapeutic agent to be reconstituted and activated. Similarly, expansion may cause loss of shell integrity, resulting in simultaneous reconstitution and release of the freeze-dried therapeutic from the shell. Encapsulation of freeze-dried therapeutic agents may provide the particles with more than one advantage over particles comprising encapsulated therapeutic agents that are not freeze-dried. For example, encapsulated freeze-dried antibodies or antibody fragments may increase shell life of the provided particles; may prolonging stability of the antibody or antibody fragment; may prolong activity of the antibody or antibody fragment; provide a clinician with increased control over how and when to deliver the antibody or antibody fragment; or combinations thereof.

In various embodiments, the provided particles are adapted to migrate to the targeted ocular tissue after administration to the subject and to deliver at least one therapeutic agent in a controllable process. In some embodiments, a therapeutic agent is coupled to the particle shell, said agent being adapted for targeted delivery. In some embodiments, the shell of the provided particles may be adapted to allow diffusion and dissolution of encapsulated therapeutic agents without expansion of the particle, and in such embodiments, the therapeutic agent is delivered at a first rate over a first period of time, the rate being tunable by selection of shell properties. In some embodiments, the first rate is slow, allowing for slow release of therapeutic agents. For example, 5-20% of therapeutic agents may be released within the first 10 days after administration. One of skill in the art will understand that numerous slow-release particle shells and methods of making the same are known. Moreover, one of skill in the art will appreciate that the first rate may change as the shell is eroded over time. In some embodiments, the shell of the provided particles may be adapted to prevent or inhibit diffusion and dissolution of encapsulated therapeutic agents until expansion of the particle, and in such embodiments, the first rate of delivery will be zero or a low rate. For example, 0-5% of therapeutic agents may be released within 10 days after administration.

The provided particles are also adapted to expand upon exposure to energy from at least one energy source. However, because exposure to energy is optional, expansion of the particle is optional. This provides clinicians with control over whether or not to expand the particles. Moreover, because energy may be applied in an iterative, controlled manner, clinicians also have control over the degree of particle expansion. Energy suitable for expansion of the provided particles may be selected from thermal energy, electromagnetic energy, and sound energy. For example, thermal energy may be delivered by a laser source, electromagnetic energy may be delivered by a microwave or a radiofrequency applier, and sound energy may be delivered by an acoustic wave generator or an ultrasound pulser. In some embodiments, the energy may be selected from ultrasound, visible light, and radio waves. Good results have been achieved with ultrasound at a frequency level of 1 MHz. Because expansion of the particle decreases shell integrity and thickness, expansion increases the rate at which the encapsulated at least one therapeutic agent is delivered. Thus, in addition to undergoing shell erosion by processes known to occur with conventional drug delivery systems, the provided particles offer a clinician the option to selectively degrade the shell by expansion, thereby providing additional control over delivery of therapeutic agents. In some embodiments, the particles may be expanded such that shell thickness and integrity are decreased (as compared to an unexpanded particle) but the shell is not compromised, which allows for diffusion of the therapeutic through the shell at a second rate that is greater than the first rate (i.e. of unexpanded state). There may be more than one delivery rate of an expanded but uncompromised particle because expansion of the particle may occur more than once, providing progressively lowered shell integrity and thickness (and progressively increased delivery rate). In some embodiments, a provided particle may be expanded to degree at which shell integrity is compromised, thereby allowing for sudden release of the therapeutic agent from the shell. In some embodiments, a provided particle may be expanded to a degree that decreases shell thickness and integrity without compromising integrity, followed by expansion to a degree that compromises shell integrity and provides delivery of therapeutic agent at a third rate, the third rate greater than the second rate.

As one of skill in the art will appreciate, the provided particles are adapted to deliver therapeutic agents in an iterative, controllable process. Such control may allow for delivery of therapeutic agents over an extended period of time. For example, it is contemplated that delivery may occur over a period of from 1 to 30 days; alternatively, from 1 to 3 months; alternatively from 1 to 4 months; alternatively, from 1 to 5 months; alternatively, from 1 to 6 months; alternatively, from 6 to 12 months. In addition to being adapted to deliver therapeutic agents in a controllable manner, the provided particles are adapted to provide a variety of options for detection and imaging of the particles before, during, and after delivery of therapeutic agents. Thus, the provided multi-functional biodegradable particles provide clinicians with selectable targeting, imaging, and therapeutic delivery options in the treatment of an ocular disorder.

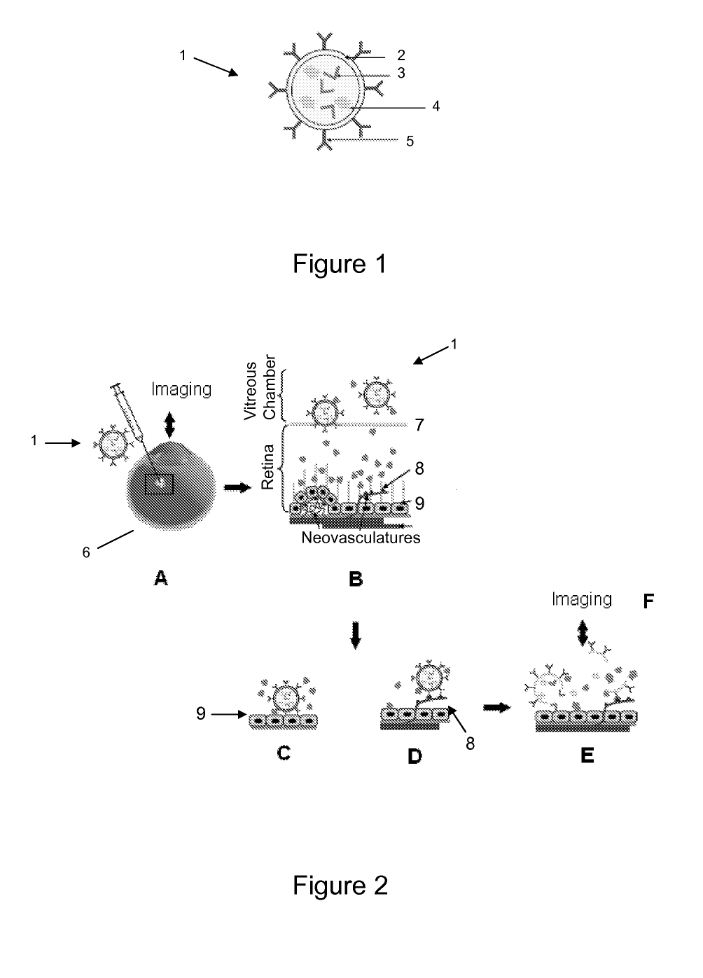

Referring to FIG. 1, one exemplary nanoparticle 1 for use in targeted treatment of AMD comprises a PLGA shell 2, encapsulated anti-VEGF antibody (therapeutic agent) 3, encapsulated indocyanine green (imaging agent) 4, and anti-VEGF R2 antibody (targeting agent) 5 coupled to the exterior surface of the shell 2. The anti-VEGF antibody may be freeze-dried. One contemplated mechanism of action of targeted delivery of therapeutic agents for treatment of AMD is illustrated in FIG. 2. When A the particles 1 described in FIG. 1 are administered by intravitreal injection into the eye 6, B they settle on the internal limiting membrane 7 separating the vitreous chamber and retina and undergo transretinal movement to C accumulate on the retinal pigment epithelium 9 and D bind with VEGF R2 receptors 8. Thereafter, E the particles 1 undergo erosion (and/or degradation by expansion) and sustained release of encapsulated anti-VEGF antibody 3 occurs, the process of which may F optionally be monitored by imaging (such as by fluorescence imaging).

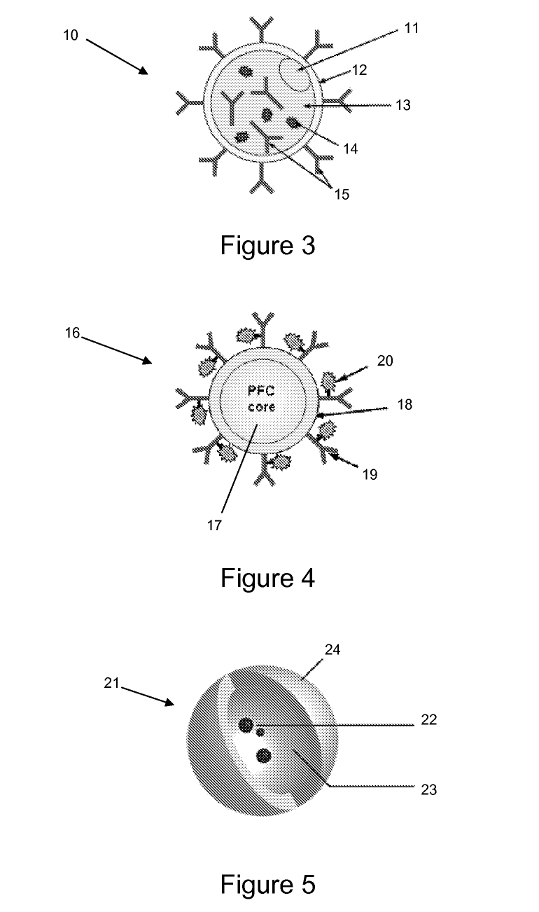

Another exemplary nanoparticle is illustrated in FIG. 3, the nanoparticle 10 comprising a perfluorocarbon droplet 11 encapsulated within a PLGA shell 12 along with phosphate buffered saline (PBS) 13, an imaging agent (such as a fluorophore) 14, and a therapeutic agent (such as the anti-VEGF agent bevacizumab) 15. As illustrated, the therapeutic agent 15 is also coupled to the exterior surface of the PLGA shell 12.

Referring to FIG. 4 another exemplary particle 16 comprises a perfluorocarbon core 17 encapsulated within a PLGA shell 18, wherein coupled to the external surface of the shell 18 are a targeting agent 19 and a fluorophore 20.

An additional exemplary particle is illustrated in FIG. 5, the particle 21 comprising India ink 22 and air 23 encapsulated within a PLGA shell 24.

### Methods of Preparing Particles

In various embodiments, provided are methods of making the provided microparticles and nanoparticles. One of skill in the art will appreciate that various properties of the particles will depend upon, among other factors, the physical properties and composition of the materials being used to construct the particles, the method of preparing the particles, and the processing conditions. In an exemplary embodiment, absorption and scattering properties of the particles depend, at least in part, upon the method of fabrication. For example, the rate of encapsulation rate of India ink into particles affects the absorption coefficient of the ink-loaded particles, which affects the absorption contrast of the particles in optical and photoacoustic imaging. As another example, the size and efficiency of filler agent encapsulation affects the scattering coefficient of the particles, which affects imaging contrast in ultrasound imaging and optical coherence tomography. Thus, particle size, shape, morphology, therapeutic release rates, erosion rate, degradation properties, and imaging properties may vary. Accordingly, the exemplary embodiments provided herein are not meant to be limiting.

In one exemplary embodiment, the provided particles may be prepared using a double emulsion method comprising (i) forming a water-in-oil (w/o) emulsion by adding an aqueous solution of at least one therapeutic agent and at least one filler agent to an organic solution of shell material and surfactant, followed by sonication or homogenization; (ii) adding the resultant emulsion with stirring into a large-volume of water containing emulsifier to form a water-in-oil-in-water (w/o/w) emulsion; (iii) removing organic solvent from the resultant emulsion by an evaporation or extraction process to form particles loaded with filler agent and therapeutic agent; and (iv) washing, centrifuging, and collecting the resultant particles. The collected particles may be lyophilized (i.e., freeze-dried).

In another exemplary embodiment, the provided particles may be prepared using an electrohydrodynamic method. Electrohydrodynamic spraying is a physical process caused by an electric force applied to the surface of liquid in an electrical field of high voltage. The electrical shear stress elongates the core and the shell liquid meniscuses formed at the outlet of co-axial capillary needles to form a cone-shaped jet which deforms and disrupts into droplets because of the electrical and mechanical forces. The electrohydrodynamic spraying process may extend the droplet sizes available from conventional mechanical atomizers to the lower range of hundred nanometers.

Characterization of prepared particles may be achieved by various known methods. For example, particle morphology may be characterized with a scanning electron microscope (such as a Hitachi S-3000 SEM). As another example, size distribution of prepared particles may be characterized by a dynamic laser scattering instrument (such as Brookhaven BI-200SM). Furthermore, surface charge of the particles may be characterized by measuring Zeta potential using a Dynamic Light Scattering device.

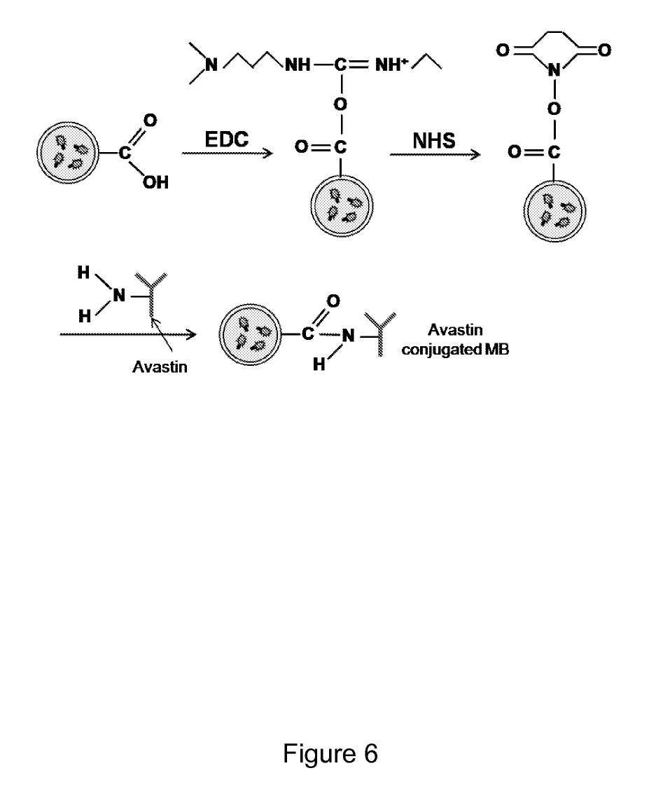

FIG. 6 schematically illustrates one exemplary embodiment of a method of conjugating a targeting agent (such as Avastin®) to a provided particle, the method comprising comprises treating carboxylic acid groups existing on the particle PLGA shell with 1-ethyl-3-(3-dimethylaminopropyl) carboiimide hydrochloride (EDC), followed by N-hydroxy succinimide (NHS) and Avastin.

### Methods of Using Particles in the Treatment of Ocular Disorders

In various embodiments, provided are methods of using the provided microparticles and nanoparticles in the treatment of an ocular disorder, said methods allowing for selectable control of targeting, imaging, and delivery of therapeutic agents. The provided methods comprise: (I) administering to a subject having an ocular disorder a composition comprising at least one provided particle, the particle comprising at least one therapeutic agent and adapted to target at least one ocular tissue and to expand upon exposure to energy from at least one energy source; (II) pausing for a pre-determined period of time to allow the administered particles to migrate to the targeted ocular tissue; (III) optionally, confirming migration to the ocular tissue using one or more ocular imaging tools; and (IV) optionally, administering to the particle sufficient energy from at least one energy source to cause the migrated particles to expand. Should administration of energy and resulting expansion of particles occur, it may optionally occur more than once.

A composition comprising provided particles may be administered to a subject via intra-ocular injection, for example, by intravitreal injection. In some embodiments, the methods comprise administering the composition (and particles contained therein) with image guidance. For example, if the administered particle comprises a fluorescence imaging agent coupled to the exterior surface of the shell, fluorescence imaging may be used to visualize administration of the particles. After administration, the particles are allowed to migrate to the targeted ocular tissue. The pre-determined period of time to allow the administered particles to migrate to the targeted ocular tissue is variable, depending at least upon the nature of the targeted tissue, the particle design, and the concentration of particles administered. For example, a period of time may be from 1-24 hours; alternatively, from 1-4 days. In an exemplary embodiment, nanoparticles targeted to the retina pigment epithelium may require from 12-24 hours to migrate through the vitreous chamber after intravitreal injection, settle on the internal limiting membrane, and accumulate on the retina pigment epithelium through pathways of transretinal movement and targeted binding. An administered nanoparticle comprising an anti-VEGF R2 antibody as the targeting agent may accumulate on the retina pigment epithelium by a process comprising targeted binding with VEGF R2 receptors on epithelial cells.

The provided methods comprise (I) administering a composition comprising at least one provided particle; (II) pausing for a pre-determined period of time to allow the administered particles to migrate to the targeted ocular tissue; and (III) confirming migration to the ocular tissue using one or more ocular imaging tools. Suitable ocular imaging tools include, but are not limited to, ultrasound imaging, optical coherence tomography, hyperspectral imaging, fluorescence imaging, and photoacoustic tomography. Ocular imaging methods and tools are well known in the art, and one of skill in the art will appreciate that the imaging method selected will depend, in part, upon the design of the particle administered. For example, fluorescence imaging would not be selected unless the particle comprises a fluorescence imaging agent. Similarly, photoacoustic tomography would not be selected unless the administered particle comprises a photoacoustic imaging agent. In some embodiments of the provided methods, migration of the administered particles is confirmed using one or more of ultrasound imaging, optical coherence tomography, photoacoustic imaging, and fluorescence imaging. In some embodiments, migration is confirmed using a combination of ultrasound imaging and fluorescence imaging. In some embodiments, migration is confirmed using only fluorescence imaging. Accordingly, the provided methods provide a clinician with options as to whether or not to confirm migration of the administered particles to the targeted ocular tissue, as well as options as to how confirmation may be achieved.

The provided methods additionally allow clinicians to control delivery of the at least one therapeutic agent in an iterative manner. The shell of the administered particle may be adapted to allow diffusion and dissolution of therapeutic agents before (or without) expansion of the administered particle. Accordingly, in some embodiments, the provided methods comprise (I) administering the particle; (II) pausing to allow for migration to the targeted ocular tissue; and (III) allowing the at least one therapeutic agent to be is delivered (via diffusion and dissolution) at a first rate over a first period of time, the rate tunable by selection of shell properties. In some embodiments, the first rate is slow. One of skill in the art will appreciate that the first rate may change as the shell is eroded over time. The shell of the administered particle may also be adapted to prevent or inhibit diffusion and dissolution of therapeutic agents until expansion of the particle, and in such embodiments, the first rate of delivery will be zero or a low rate.

In some embodiments, the provided methods comprise (I) administering the particle; (II) pausing to allow for migration to the targeted ocular tissue; and (III) administering sufficient energy from at least one energy source to expanded the particle. The energy type and energy source may be dictated by particle design or may be selected by a clinician to meet one or more specific needs. Energy suitable for expansion of the provided particles may be selected from thermal energy, electromagnetic energy, and sound energy. For example, the energy may be selected from ultrasound, visible light, and radio waves. Good results have been achieved with ultrasound. In some embodiments, the selected energy may be administered in a manner allowing for iterative, controlled expansion of the particle. Because expansion of the particle decreases shell integrity and thickness, expansion increases the rate at which the at least one therapeutic agent is delivered.

In some embodiments, the provided methods comprise administering the energy in a manner (determined by selection of one or more of energy type, energy source, energy strength, and duration of exposure) that expands the particle such that shell thickness and integrity are decreased (as compared to an unexpanded particle) but the shell is not compromised. This allows for diffusion of the therapeutic through the shell at a second rate that is greater than the first rate (of unexpanded particle). In some embodiments, the provided methods comprise administering the energy in the described manner such that expansion of the particle occurs more than once. Administration in this manner progressively lowers shell integrity and thickness, which progressively increases delivery rate of the at least one therapeutic agent.

In some embodiments, the provided methods comprise administering the energy in a manner that expands the particle to a degree at which shell integrity is compromised, thereby allowing for sudden release of the at least one therapeutic agent from the particle. Thus, in such embodiments, delivery rate of the at least one therapeutic agent will be greater than that of the unexpanded particle (first rate), as well as greater than that of any particle previously expanded to a lesser degree (i.e., without compromising shell integrity). One of skill in the art will appreciate that any expansion of an administered particle may also only be to a degree that compromises shell integrity.

In various embodiments, the provided methods further comprise using one or more ocular imaging tools to detect and image the administered particles during delivery of the at least one therapeutic agent, after delivery of the at least one therapeutic agent, or both. Accordingly, particles may be detected and imaged before; during; after; before and during; before and after; during and after; or before, during, and after delivery of the at least one therapeutic agent. One of skill in the art will appreciate that the imaging tool selected will depend, in part, upon the design of the particle administered. In some embodiments, the particle may comprise an encapsulated imaging agent, such as a fluorescence imaging agent. Accordingly, fluorescence imaging would be an appropriate tool for determining particle concentration and localization during delivery of therapeutic agents. In some embodiments, particle expansion and therapeutic agent delivery are monitored using one or more of ultrasound imaging, optical coherence tomography, photoacoustic imaging, and fluorescence imaging. In some embodiments, monitoring is by a combination of ultrasound imaging and fluorescence imaging.

In an exemplary embodiment of the provided methods, (I) a composition comprising at least one provided nanoparticle comprising at least one anti-VEGF R2 antibody targeting agent is administered to a subject having AMD; (II) the administered nanoparticles are provided with a pre-determined period of time to migrate to the retina pigment epithelium; (III) optionally, migration to the retina pigment epithelium is confirmed using one or more ocular imaging tools; and (IV) sufficient energy to cause the migrated nanoparticles to expand is administered using at least one energy source. In the exemplary embodiment, the administered nanoparticle may comprise a freeze-dried AMD therapeutic agent (such as bevacizumab). In an exemplary embodiment, the administered nanoparticle comprises a freeze-dried anti-VEGF antibody and the particle shell is adapted to prevent or inhibit diffusion and dissolution prior to expansion of the nanoparticle. Accordingly, the clinician has a variety of options as to how to deliver the therapeutic agent to treat AMD. It may be delivered by sudden release if the clinician chooses to administer the energy in a manner that expands the nanoparticle to a degree that compromises shell integrity. It may be delivered at a slower rate if the clinician chooses to administer the energy in a manner that expands the nanoparticle to a degree that decreases shell thickness and integrity but does not compromise shell integrity. Moreover, it may be delivered at progressively increased rates if the clinician chooses to administer the energy more than once, each exposure decreasing shell thickness. Thus, a clinician may use the provided methods to deliver AMD therapeutics in an iterative, controlled process. Additionally, a clinician may also choose to monitor nanoparticle expansion and therapeutic agent delivery using one or more ocular tools. For example, a clinician may utilize ultrasound imaging and fluorescence imaging to monitor the delivery of bevacizumab. Thus, the provided methods and nanoparticles provide for real-time imaging of therapeutic response and seamless integration of clinical imaging and therapy.

In practice of the provided methods, the particles may be administered by a clinician (doctor or other medical personnel) at a hospital or other medical facility in accordance with acceptable medical practice, taking into account the condition of the subject, including injection site condition, patient age, sex, body weight, and other determinable factors. The “effective amount” administered for purposes herein is thus determined by such considerations, as is known in the art, and is of sufficient amount to achieve the desired response, including but not limited to, an amount sufficient to achieve improvement or elimination of symptoms of the disorder. The provided methods may be practiced on mammals, including humans.

In the practice of the provided methods, the composition comprising the provided particles may be an aqueous solution. In addition to the particles, it may also comprise one or more other pharmaceutically active ingredients, pharmaceutically acceptable carriers, diluents, adjuvants, vehicles, or combinations thereof. Pharmaceutically acceptable carriers, diluents, adjuvants and vehicles generally refer to inert, non-toxic solid or liquid fillers not reactive with the particles or any pharmaceutically active ingredient present in the composition.

In practice of the provided methods, the composition comprising the particles may be administered as a single dose or multiple doses over a period of days. The compositions will generally be formulated in a pharmaceutically acceptable unit dosage form. Examples of pharmaceutically acceptable unit dose form include, but are not limited to, sterile solutions, suspensions, and dispersions in a carrier, or sterile powders capable of being reconstituted with a carrier into sterile solutions, suspensions, and dispersions. Suitable carriers may comprise, for example, water, ethanol, polyol (such as glycerol, propylene glycol, or liquid polyethylene glycol), vegetable oils, and combinations thereof.

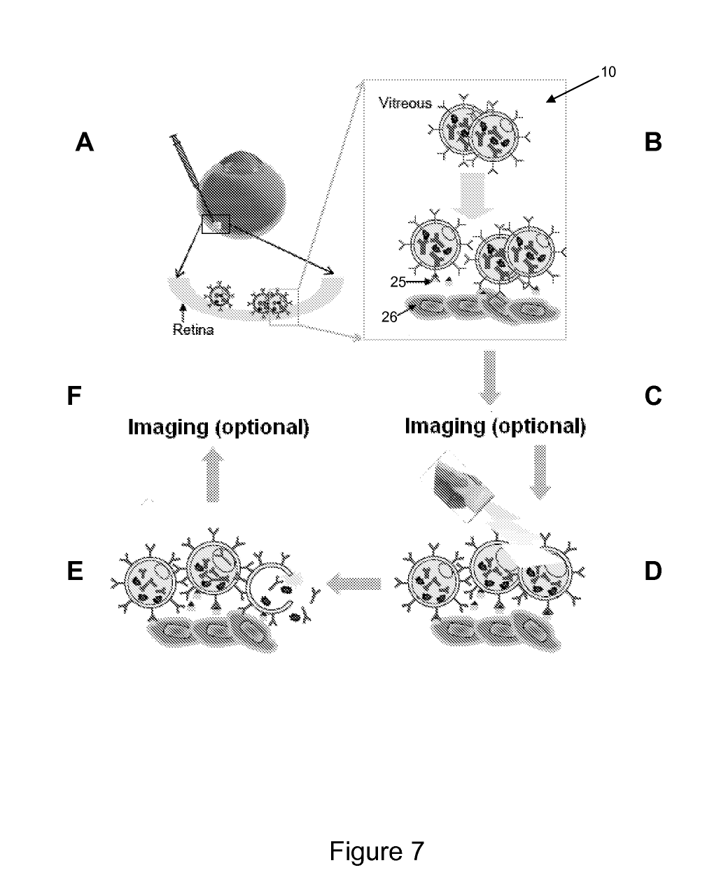

An exemplary method of using the provided particles to treat an ocular disorder is illustrated in FIG. 7, the method comprising: A administering nanoparticles 10 (described in FIG. 3) by intravitreal injection; B allowing a sufficient period of time for the particles 10 to target VEGF 25 and diffuse to and accumulate at the retinal pigment epithelium 26; C optionally, using ocular imaging tools to monitor the accumulation of the particles 10 at the retinal pigment epithelium 26; D administering high intensity ultrasound pulses to expand the particles 10; E allowing the particles to erode (or degrade) and release the encapsulated agents, a process that F may optionally be monitored by imaging tools (such as fluorescence imaging).

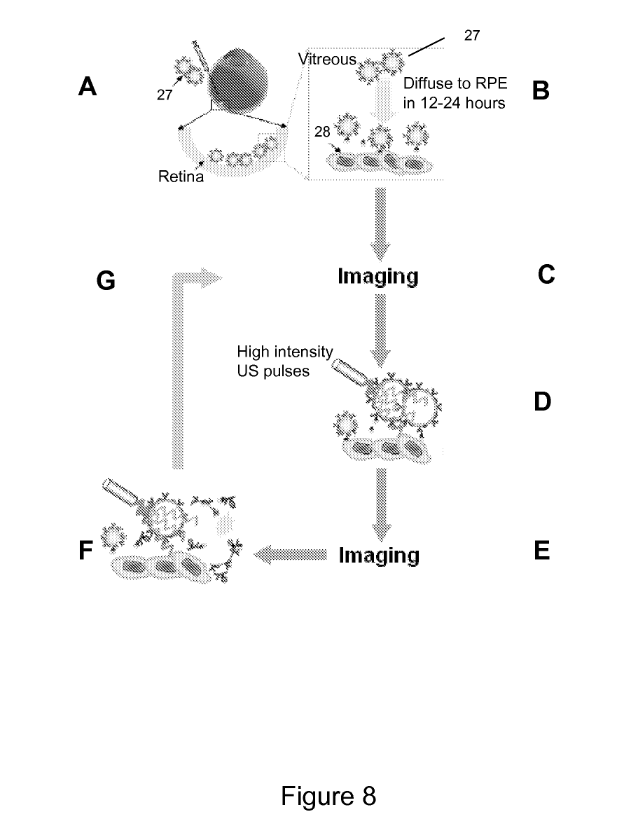

Referring to FIG. 8, illustrated is one exemplary method for image-guided delivery of anti-VEGF therapeutics for the treatment of AMD. The method comprises A administering nanoparticles 27 by intravitreal injection; B allowing 12-24 hours for the administered particles 27 to diffuse to and accumulate at the retinal pigment epithelium 28; C using imaging tools (such as a combination of fluorescence imaging and ultrasound imaging) to monitor the accumulation of the nanoparticles 27 and any pre-expansion shell erosion or therapeutic agent diffusion; D administering high intensity ultrasound pulses to expand the particles 27; E using imaging tools (such as ultrasound imaging) to monitor nanoparticle 27 erosion and therapeutic agent diffusion; F administering high intensity ultrasound pulses to further expand the particles 27 and degrade them such that the rate of diffusion is increased or the encapsulated agents are released from the shell; and G using imaging tools (such as fluorescence imaging) to monitor therapeutic agent diffusion; wherein steps C, D, E, F, and G may be repeated over a period of days, weeks, or months.

### EXAMPLES

The described embodiments will be better understood by reference to the following examples which are offered by way of illustration not limitation.

### Example 1

### Preparation of PLGA Particles Encapsulating Perfluorocarbon Filler Agent

PLGA (25 mg) (Boehringer Ingelheim) is dissolved in 2 mL methylene chloride (Fisher Scientific), followed by addition of 1 mL of methylene chloride to the resulting solution and mixing. 20 mL of 1.5 w/v % sodium cholate is mixed with the solution, and the final mixture emulsified at 20,000 rpm for 2 minutes and stirred by a magnetic bar for three hours to evaporate the methylene chloride.

The resulting emulsion is centrifuged at 3000 rpm for 5 minutes, and the precipitated PLGA particles collected and re-dispersed by deionized water. Centrifugation and re-dispersion is repeated three times, and the size distribution and zeta potential of the resulting particles characterized by dynamic light scattering (DLS) and electrophoretic light scattering (ELS) techniques, respectively.

### Example 2

### Coupling of PLGA Particle Shells with Fluorescence Imaging Agent and Targeting Antibody

2 mg of PLGA particles prepared according to Example 1 are dispersed into 1 mL 0.1M MES [2-(N-morpholino)ethanesulfonic acid] buffer solution with pH 5-6 (Sigma Aldrich). 1-ethyl-3-(3-dimethylaminopropyl) carbodiimide hydrochloride (EDC) (Sigma Aldrich) and N-hydroxy succinimide (NHS) (Sigma Aldrich) are consecutively added to the particle suspension such that the final concentration of EDC is 2 mM and the final concentration of NHS is 5 mM. The resulting mixture of NHS-ester active particles is stirred for 20 minutes and then centrifuged and washed with MES (pH 5-6) three times to remove extra EDC and NHS. To conjugate bevacizumab, 0.1 M MES solution (pH 7-8) is added to the NHS-ester activated particles and dispersed well, followed by addition of bevacizumab. The resulting mixture is stirred for 2 hours, centrifuged, and washed with PBS three times. The particles may be re-suspended in 1 M sodium bicarbonate buffer (pH 9).

A fluorescence imaging agent may be coupled to bevacizumab, which has multiple binding sites. Alexa Fluor 680 N-succinimidyl ester is added to the suspension of particles and stirred for 1 hour at room temperature, followed by elution in a PD-10 desalting column for purification.

### Example 3

### Preparation of PLGA Particles Comprising Ranibizumab Using a Double Emulsion Method

An aqueous solution of Indocyanine Green (0.5 mM), ranibizumab (10% w/v), and PVA (1% w/v) is emulsified in a 3% (w/v) PLGA/chloroform solution by a homogenizer. The primary emulsion is added dropwise to a 2% (w/v) PVA solution and further emulsified. The double emulsion is then added to a 5% isopropanol solution, centrifuged, and the particle precipitate collected, filtered, and washed.

The particles are dispersed in MES buffer, and EDC and NHS are consecutively added, followed by stirring and washing with MES buffer to obtain a suspension of NHS-ester activated particles. An anti-VEGF R2 antibody may be surface coupled by adding it to the NHS-ester active particle suspension and stirring the mixture for 2 hours, centrifuging, and washing with PBS three times. The particles may be re-suspended in 1 M sodium bicarbonate buffer (pH 9).

### Example 4

### Method of Preparing PLGA Particles Using Electrohydrodynamic Spray Technique

The following solutions are prepared as described:

1. Solution A is prepared by placing desired amount (typically 60-300 mg) of PLGA into a 15 mL tube, adding 3 mL CH₂Cl₂ in the tube, and mixing. The amount of PLGA used determines the final particle size.

2. Solution B is prepared by placing the desired amount of material to be encapsulated (antibody, fluorescence imaging agent, etc.) into 1.5 mL of distilled water and mixing. The amount of material used depends upon the intended application.

3. Solution C is prepared by placing 20 mL ethanol into a collection beaker.

4. Solution D is prepared by placing 2.5 g of polyvinyl alcohol (PVA) into 50 mL of distilled water and mixing. If necessary, heating may be necessary to mix.

Solution A and Solution B are placed into separate 3 mL syringes that are attachable to an infusion pump suitable for use with electrohydrodynamic spray techniques. A flat gauge 28 needle is positioned inside of a flat gauge 14 needle, and the Solution A syringe is connected with the gauge 14 needle and the Solution B syringe is connected with the gauge 28 needle. The two needles are fixed on an infusion pump having 0.5-60 mL/hr flow rate. The flow rate of the Solution A/gauge 14 needle is adjusted to be at 10 mL/h, and the flow rate of the Solution B/gauge 28 needle is adjusted to be at 5 mL/h. However, different flow rates (such as from 2 mL/hr to 20 mL/hr) may be used to modify particle diameter and shell thickness. A circular electrode with 15 cm diameter is fixed 12 mm below the co-axial syringe tip, which is adjusted to be at the center of the electrode. The syringes are connected to a positive power supply (adjusted to 4 kV) and the electrode is connected with a negative power supply (adjusted to −1 kV).

Solution C is placed below the circular electrode, the power supplies are turned on, and the infusion pump is turned on and allowed to flow, wherein the resulting particles are collected in Solution C.

The collected particles (in Solution C) are mixed with Solution D for 3 hours and then centrifuged as follows: (i) once at 2000 rpm for 7 minutes (keep residual and dissolve in water using Vortex mixer); (ii) twice at 300 rpm for 7 minutes (keep clean portion); (iii) 3-6 times at 2000 rpm for 7 minutes (keep residual and dissolve in water using Vortex mixer); and (iv) once at 2000 rpm for 7 minutes (keep residual and dissolve in water using Vortex mixer).

The final centrifuged solution comprising the particles may be lyophilized, and the particles characterized using scanning electron microscopy, laser scattering, microscopy, or other combinations thereof.

The present invention should not be considered limited to the specific examples described herein, but rather should be understood to cover all aspects of the invention. Various modifications, equivalent processes, as well as numerous structures and devices to which the present invention may be applicable will be readily apparent to those of skill in the art. Those skilled in the art will understand that various changes may be made without departing from the scope of the invention, which is not to be considered limited to what is described in the specification.

What is claimed is:

1. A biodegradable particle for targeted delivery and controlled release of at least one agent for the treatment of an ocular disorder of a subject, comprising:
(a) a biodegradable shell comprising an exterior surface;
(b) at least one ocular targeting agent coupled to the exterior surface of the shell;
(c) at least one filler agent encapsulated within the shell; and
(d) at least one therapeutic agent for treatment of the ocular disorder encapsulated within the shell;
wherein the particle is adapted to migrate to the ocular target after administration to the subject and (i) deliver the at least one therapeutic agent at a first rate over a first pre-determined period of time; (ii) optionally, expand upon exposure to energy from at least one energy source and deliver the at least one therapeutic agent at a second rate over a second pre-determined period of time, the second rate greater than the first rate; and (iii) optionally, expand upon exposure to energy from at least one energy source and deliver the at least one therapeutic agent at a third rate over a third pre-determined period of time, the third rate greater than the second rate.

2. A particle according to claim 1, wherein the ocular disorder is selected from age-related macular degeneration, glaucoma, infective conjunctivitis, allergic conjunctivitis, ulcerative keratitis, non-ulcerative keratitis, episcleritis, scleritis, diabeticretinopathy, uveitis, endophthalmitis, infectious conditions, and inflammatory conditions.

3. A particle according to claim 2, wherein the ocular disorder is age-related macular degeneration.

4. A particle according to claim 3, wherein the ocular target is retina pigment epithelium and the targeting agent is anti-VEGF R2 antibody.

5. A particle according to claim 1, wherein the shell comprises a material selected from lipid, human serum albumin, polylactide (PLA), poly(ε-caprolactone) (PCL), poly(glycolic acid) (PGA), poly(lactide-co-glycolide) (PLGA), chitosan, eudragit, hyaluronic acid, alginate, carboxy methyl cellulose, carbopol, polyethylene glycol, poly(ethyl-2-cyanoacrylate) (PECA), polystyrene, poloxamers, Hydroxypropyl Methylcellulose (HPMC), 2-hydroxyethyl methacrylate (HEMA), polyvinyl alcohol (PVA), poly(methyl acrylate) (PMA), and combinations thereof.

6. A particle according to claim 1, wherein the filler agent is selected from air, perfluorocarbon, nitrogen, saline, phosphate buffered saline, water, fluorescence imaging agent, photoacoustic agent, and combinations thereof.

7. A particle according to claim 6, wherein the photoacoustic agent is selected from India ink, methylene blue, gold nanoparticles, carbon nanotubes, Fiesta Red, and Rhodamine.

8. A particle according to claim 6, wherein the fluorescence imaging agent is selected from indocyanine green, cyanine 5, cyanine 7, cyanine 9, Texas Red, Nile Red, fluorescein, green fluorescent protein, red fluorescent protein, yellow fluorescent protein, IRDye™ 800CW, near infrared fluorescence type II quantum dots, fluorescent beads, AlexaFluor™ 680, and combinations thereof.

9. A particle according to claim 8, comprising perfluorocarbon and a fluorescence imaging agent.

10. A particle according to claim 9, wherein the perfluorocarbon is selected from liquid perfluorocarbon and gaseous perfluorocarbon.

11. A particle according to claim 1, comprising an imaging agent coupled to the exterior surface of the shell.

12. A particle according to claim 11, wherein the imaging agent is a fluorescence imaging agent selected from indocyanine green, cyanine 5, cyanine 7, cyanine 9, Texas Red, Nile Red, fluorescein, green fluorescent protein, red fluorescent protein, yellow fluorescent protein, IRDye™ 800CW, near infrared fluorescence type II quantum dots, fluorescent beads, AlexaFluor™ 680, and combinations thereof.

13. A particle according to claim 1, wherein the therapeutic agent is selected from bevacizumab, ranibizumab, pegabtanib, oligonucleotides, Acetazolamide, Pilocarpine HCl, Insulin, Cyclopentolate, Timolol maleate, GCV, Pilocarpine, Amikacin, Flurbiprofen, Cyclosporin, Rhodamine, Dexamethasone, Pilocarpine nitrate, tripicamide, antibiotics, antifungal agents, anti-viral agents, and combinations thereof.

14. A particle according to claim 13, wherein the therapeutic agent is freeze-dried.

15. A particle according to claim 1, wherein the energy is ultrasound.

16. A particle according to claim 1, wherein the particle is a microparticle.

17. A particle according to claim 1, wherein the particle is a nanoparticle.

18. A nanoparticle for targeted delivery and controlled release of at least one agent for the treatment of age-related macular degeneration in a subject, comprising:
(a) a biodegradable shell comprising an exterior surface;
(b) at least one anti-VEGF R2 antibody and optionally, at least one imaging agent, coupled to the exterior surface of the shell;
(c) at least one filler agent encapsulated within the shell, the filler agent selected from air, liquid perfluorocarbon, gaseous perfluorocarbon, nitrogen, saline, phosphate buffered saline, water, photoacoustic agent, fluorescence imaging agent, and combinations thereof; and
(d) at least one therapeutic agent for treatment of age-related macular degeneration encapsulated within the shell;
wherein the nanoparticle is adapted to be detected by one or more ocular imaging tools; and
wherein the nanoparticle is adapted to migrate to the retina pigment epithelium after intravitreal injection into the subject and (i) deliver the at least one therapeutic agent at a first rate over a first pre-determined period of time; (ii) optionally, expand upon exposure to energy from at least one energy source and deliver the at least one therapeutic agent at a second rate over a second pre-determined period of time, the second rate greater than the first rate; and (iii) optionally, expand upon exposure to energy from at least one energy source and deliver the at least one therapeutic agent at a third rate over a third pre-determined period of time, the third rate greater than the second rate.

19. A nanoparticle according to claim 18, wherein the shell comprises a material selected from lipid, human serum albumin, polylactide (PLA), poly(ε-caprolactone) (PCL), poly(glycolic acid) (PGA), poly(lactide-co-glycolide) (PLGA), chitosan, eudragit, hyaluronic acid, alginate, carboxy methyl cellulose, carbopol, polyethylene glycol, poly(ethyl-2-cyanoacrylate) (PECA), polystyrene, poloxamers, Hydroxypropyl Methylcellulose (HPMC), 2-hydroxyethyl methacrylate (HEMA), polyvinyl alcohol (PVA), poly(methyl acrylate) (PMA), and combinations thereof.

20. A nanoparticle according to claim 18, comprising at least one imaging agent coupled to the shell, the imaging selected from indocyanine green, cyanine 5, cyanine 7, cyanine 9, fluorescein, and green fluorescent protein.

21. A nanoparticle according to claim 18, wherein the at least one filler agent is liquid perfluorocarbon, gaseous perfluorocarbon, fluorescence imaging agent, photoacoustic imaging agent, or combinations thereof.

22. A nanoparticle according to claim 18, wherein the therapeutic agent is selected from bevacizumab, ranibizumab, pegabtanib, and combinations thereof.

23. A nanoparticle according to claim 22, wherein the therapeutic agent is freeze-dried.

24. A method for targeted delivery and controlled release of at least one therapeutic agent to ocular tissue of a subject having an ocular disorder, comprising:
(I) administering to the subject a composition comprising at least one biodegradable particle adapted to target at least one ocular tissue, each particle comprising:
(a) a biodegradable shell comprising an exterior surface;
(b) at least one ocular targeting agent and optionally, at least one imaging agent, coupled to the exterior surface of the shell;
(c) at least one filler agent encapsulated within the shell; and
(d) at least one therapeutic agent for treatment of the ocular disorder encapsulated within the shell;
wherein the particle is adapted to expand upon exposure to energy from at least one energy source;
(II) pausing for a pre-determined period of time to allow the administered particles to migrate to the ocular target;
(III) optionally, confirming migration to the ocular target using one or more ocular imaging tools; and
(IV) optionally, administering sufficient energy from at least one energy source to cause the migrated particles to expand;
wherein the at least one therapeutic agent is delivered (i) at a first rate over a first pre-determined period of time; (ii) optionally, at a second rate over a second pre-determined period of time upon expansion of the particles by exposure to energy from at least one energy source, the second rate greater than the first rate; and (iii) optionally, at a third rate over a third pre-determined period of time upon expansion of the particles by exposure to energy from at least one energy source, the third rate greater than the second rate.

25. A method according to claim 24, wherein the ocular disorder is selected from age-related macular degeneration, glaucoma, infective conjunctivitis, allergic conjunctivitis, ulcerative keratitis, non-ulcerative keratitis, episcleritis, scleritis, diabeticretinopathy, uveitis, endophthalmitis, infectious conditions, and inflammatory conditions.

26. A method according to claim 25, wherein the ocular disorder is age-related macular degeneration.

27. A method according to claim 26, wherein the ocular target is retina pigment epithelium and the targeting agent is anti-VEGF R2 antibody.

28. A method according to claim 24, wherein the shell comprises a material selected from lipid, human serum albumin, polylactide (PLA), poly(ε-caprolactone) (PCL), poly(glycolic acid) (PGA), poly(lactide-co-glycolide) (PLGA), chitosan, eudragit, hyaluronic acid, alginate, carboxy methyl cellulose, carbopol, polyethylene glycol, poly(ethyl-2-cyanoacrylate) (PECA), polystyrene, poloxamers, Hydroxypropyl Methylcellulose (HPMC), 2-hydroxyethyl methacrylate (HEMA), polyvinyl alcohol (PVA), poly(methyl acrylate) (PMA), and combinations thereof.

29. A method according to claim 24, wherein the administered particles comprise at least one imaging agent coupled to the shell, the imaging selected from indocyanine green, cyanine 5, cyanine 7, cyanine 9, fluorescein, and green fluorescent protein.

30. A method according to claim 24, wherein the filler agent is selected from air, perfluorocarbon, nitrogen, saline, phosphate buffered saline, water, fluorescence imaging agent, photoacoustic agent, and combinations thereof.

31. A method according to claim 30, wherein the fluorescence imaging agent is selected from indocyanine green, cyanine 5, cyanine 7, cyanine 9, Texas Red, Nile Red, fluorescein, green fluorescent protein, red fluorescent protein, yellow fluorescent protein, IRDye™ 800CW, near infrared fluorescence type II quantum dots, fluorescent beads, AlexaFluor™ 680, and combinations thereof.

32. A method according to claim 30, wherein the photoacoustic agent is selected from India ink, methylene blue, gold nanoparticles, carbon nanotubes, Fiesta Red, and Rhodamine.

33. A method according to claim 24, wherein the therapeutic agent is selected from bevacizumab, ranibizumab, pegabtanib, oligonucleotides, Acetazolamide, Pilocarpine HCl, Insulin, Cyclopentolate, Timolol maleate, GCV, Pilocarpine, Amikacin, Flurbiprofen, Cyclosporin, Rhodamine, Dexamethasone, Pilocarpine nitrate, tripicamide, antibiotics, antifungal agents, anti-viral agents, and combinations thereof.

34. A method according to claim 33, wherein the therapeutic agent is freeze-dried.

35. A method according to claim 24, wherein the ocular imaging tool is selected from ultrasound imaging, optical coherence tomography, hyperspectral imaging, fluorescence imaging, and photoacoustic tomography.

36. A method according to claim 35, comprising visualizing expanded particles using one or more ocular imaging tools.

37. A method according to claim 24, wherein the energy is ultrasound.

38. A method according to claim 24, wherein the composition administered comprises microparticles.

39. A method according to claim 24, wherein the composition administered comprises nanoparticles.

40. A method for targeted delivery and controlled release of at least one therapeutic agent to the retina pigment epithelium of a subject having age-related macular degeneration, comprising:
(I) administering to the subject an intravitreal injection of a composition comprising at least one biodegradable nanoparticle adapted to target the retina pigment epithelium, each particle comprising:
(a) a biodegradable shell comprising an exterior surface;
(b) at least one anti-VEGF R2 antibody and optionally, at least one imaging agent, coupled to the exterior surface of the shell;
(c) at least one filler agent encapsulated within the shell, the filler agent selected from air, liquid perfluorocarbon, gaseous perfluorocarbon, nitrogen, saline, phosphate buffered saline, water, photoacoustic agent, fluorescence imaging agent, and combinations thereof; and
(d) at least one therapeutic agent for treating age-related macular degeneration encapsulated within the shell;
wherein the particle is adapted to expand upon exposure to energy from at least one energy source;
(II) pausing for a pre-determined period of time to allow the administered nanoparticles to migrate to the retina pigment epithelium;
(III) optionally, confirming migration to the retina pigment epithelium using one or more ocular imaging tools; and
(III) optionally, administering sufficient energy from at least one energy source to cause the migrated nanoparticles to expand;
wherein the at least one therapeutic agent is delivered (i) at a first rate over a first pre-determined period of time; (ii) optionally, at a second rate over a second pre-determined period of time upon expansion of the particles by exposure to energy from at least one energy source, the second rate greater than the first rate; and (iii) optionally, at a third rate over a third pre-determined period of time upon expansion of the particles by exposure to energy from at least one energy source, the third rate greater than the second rate.

41. A method according to claim 40, wherein the shell comprises a material selected from lipid, human serum albumin, polylactide (PLA), poly(ε-caprolactone) (PCL), poly(glycolic acid) (PGA), poly(lactide-co-glycolide) (PLGA), chitosan, eudragit, hyaluronic acid, alginate, carboxy methyl cellulose, carbopol, polyethylene glycol, poly(ethyl-2-cyanoacrylate) (PECA), polystyrene, poloxamers, Hydroxypropyl Methylcellulose (HPMC), 2-hydroxyethyl methacrylate (HEMA), polyvinyl alcohol (PVA), poly(methyl acrylate) (PMA), and combinations thereof.

42. A method according to claim 40, wherein the administered nanoparticle comprises at least one imaging agent coupled to the shell, the imaging selected from indocyanine green, cyanine 5, cyanine 7, cyanine 9, fluorescein, and green fluorescent protein.

43. A method according to claim 40, wherein the at least one filler agent is liquid perfluorocarbon, gaseous perfluorocarbon, fluorescence imaging agent, photoacoustic imaging agent, or combinations thereof.

44. A method according to claim 40, wherein the therapeutic agent is selected from bevacizumab, ranibizumab, pegabtanib, and combinations thereof.

45. A method according to claim 44, wherein the therapeutic agent is freeze-dried.

46. A method according to claim 40, wherein the ocular imaging tool is selected from ultrasound imaging, optical coherence tomography, hyperspectral imaging, fluorescence imaging, and photoacoustic tomography.

47. A method according to claim 46, comprising visualizing expanded nanoparticles using one or more ocular imaging tools.

48. A method according to claim 40, wherein the energy is ultrasound.
